(19) United States
(12) Patent Application Publication
(10) Pub. No.: US 2025/0095857 A1
(43) Pub. Date: Mar. 20, 2025

(54) MACHINE LEARNING ENABLED PATIENT STRATIFICATION

(71) Applicant: The Regents of the University of California, Oakland, CA (US)

(72) Inventors: Shamim Nemati, San Diego, CA (US); Supreeth Prajwal Shashikumar, San Diego, CA (US); Atul Malhotra, San Diego, CA (US); Jonathan Lam, San Diego, CA (US)

(21) Appl. No.: 18/292,291

(22) PCT Filed: Jul. 29, 2022
(86) PCT No.: PCT/US2022/038926

§ 371 Date: Jan. 25, 2024

Related U.S. Application Data
(60) Provisional application No. 63/227,885, filed on Jul. 30, 2021.

(51) Int. Cl. G16H 50/30; G16H 10/60

(52) U.S. Cl. CPC G16H 50/30; G16H 10/60

(57) ABSTRACT

A method for patient stratification may include applying a first machine learning model to determine, based on a clinical data of a patient, a risk score for the patient. Where the risk score for the patient exceeds a threshold, a second machine learning model may be applied to determine a first probability of the risk score being a false positive. Where the risk score for the patient fails to exceed the threshold, a third machine learning model may be to determine a second probability of the risk score being a false negative. Clinical recommendations for the patient may be determined based on the risk score, the first probability of the risk score being the false positive, and the second probability of the risk score being the false negative. Related systems and computer program products are also provided.

### CROSS REFERENCE TO RELATED APPLICATION

This application claims priority to U.S. Provisional Application No. 63/227,885, entitled “METHODS FOR ACCURATE PATIENT STRATIFICATION USING DEEP LEARNING PREDICTIVE MODELS” and filed on Jul. 30, 2021, the disclosure of which is incorporated herein by reference in its entirety.

### STATEMENT OF GOVERNMENT SUPPORT

This invention was made with government support under ES025445 and LM013517, awarded by the National Institute of Health (NIH). The government has certain rights in the invention.

### TECHNICAL FIELD

The subject matter described herein relates generally to machine learning and more specifically to deep learning enabled techniques for patient stratification.

### BACKGROUND

In many domains, early recognition of diseases and timely initiation of medical interventions have been shown to be an effective approach to improve clinical outcomes. As one example, early recognition of life-threatening conditions, such as sepsis, as well as timely initiation of life-saving treatments in hospitalized patients, such as antibiotics, have increased patient survival.

### SUMMARY

Systems, methods, and articles of manufacture, including computer program products, are provided for machine learning enabled patient stratification. In one aspect, there is provided a system for patient stratification. The system may include at least one processor and at least one memory. The at least one memory may include program code that provides operations when executed by the at least one processor. The operations may include: applying a first machine learning model to determine, based at least on a clinical data of a patient, a risk score for the patient; in response to the risk score for the patient exceeding a first threshold, applying a second machine learning model to determine a first probability of the risk score being a false positive; in response to the risk score for the patient failing to exceed the first threshold, applying a third machine learning model to determine a second probability of the risk score being a false negative; and determining, based at least on the risk score, the first probability of the risk score being the false positive, and the second probability of the risk score being the false negative, one or more clinical recommendations for the patient.

In another aspect, there is provided a method for machine learning enabled patient stratification. The method may include: applying a first machine learning model to determine, based at least on a clinical data of a patient, a risk score for the patient; in response to the risk score for the patient exceeding a first threshold, applying a second machine learning model to determine a first probability of the risk score being a false positive; in response to the risk score for the patient failing to exceed the first threshold, applying a third machine learning model to determine a second probability of the risk score being a false negative; and determining, based at least on the risk score, the first probability of the risk score being the false positive, and the second probability of the risk score being the false negative, one or more clinical recommendations for the patient.

In another aspect, there is provided a non-transitory computer readable medium storing instructions. When executed by at least one data processor, the instructions may cause operations that include: applying a first machine learning model to determine, based at least on a clinical data of a patient, a risk score for the patient; in response to the risk score for the patient exceeding a first threshold, applying a second machine learning model to determine a first probability of the risk score being a false positive; in response to the risk score for the patient failing to exceed the first threshold, applying a third machine learning model to determine a second probability of the risk score being a false negative; and determining, based at least on the risk score, the first probability of the risk score being the false positive, and the second probability of the risk score being the false negative, one or more clinical recommendations for the patient.

In some variations of the methods, systems, and non-transitory computer readable media, one or more of the following features can optionally be included in any feasible combination. A conformity metric indicative of a similarity between the clinical data of the patient and one or more conformal sets may be determined. In response to the conformity metric satisfying a second threshold, the first machine learning model may be applied to determine the risk score for the patient. In response to the conformity metric failing to satisfy the second threshold, the clinical data of the patient may be rejected as indeterminate.

In some variations, the clinical data of the patient may be encoded to generate a reduced dimension representation of the clinical data. The conformity metric indicative of the similarity between the clinical data of the patient and one or more conformal sets may be determined based at least on the reduced dimension representation of the clinical data.

In some variations, the conformity metric may include a Euclidean distance or a cosine distance.

In some variations, the one or more conformal sets may include a control conformal set of clinical data associated with patients without a disease and a case conformal set of clinical data associated with patients with the disease.

In some variations, the one or more conformal sets may be generated by at least clustering training data including true cases of patients with a disease, true controls of patients without the disease, false positives of patients without the disease but diagnosed as having the disease, and false negatives of patients with the disease but diagnosed as without the disease.

In some variations, the first probability of the risk score being the false positive and the second probability of the risk score being the false negative may be determined based on one or more of a quantity of missing clinical variables in the clinical data, an uncertainty associated with the risk score, an extent of conformity between the clinical data and the one or more conformal sets, and a number of nearest neighbors with discordant labels or a spread in the risk score.

In some variations, an uncertainty associated with the risk score of the patient may be determined. The one or more clinical recommendations for the patient may be determined based at least on the uncertainty associated with the risk score.

In some variations, the uncertainty associated with the risk score of the patient may include an uncertainty associated with the first machine learning model. The uncertainty associated with the first machine learning model may be determined by at least applying a Monte Carlo dropout to assess a change in the risk score caused by ignoring an output of one or more layers of the first machine learning model.

In some variations, the uncertainty associated with the risk score of the patient may include an uncertainty associated with the clinical data. The uncertainty associated with the clinical data may be determined by at least assessing a change in the risk score caused by excluding random portions of the clinical data.

In some variations, the uncertainty associated with the risk score of the patient may be assessed based on a quantity similar patients with discordant labels and/or a spread in risk score determined at least by repeated substitution of at least one missing feature of the patient by a value of a corresponding feature or a most relevant feature from similar patients.

In some variations, the first machine learning model, the second machine learning model, and the third machine learning model may be feed forward neural networks.

In some variations, the one or more clinical recommendations for the patient may be determined by applying a decision tree to the risk score, an uncertainty associated with the risk score, a contextual information for the patient, the first probability of the risk score being the false positive, and the second probability of the risk score being the false negative.

In some variations, the one or more clinical recommendations may be generated based at least on a context of the patient being one of emergency, general wards, or intensive care.

In some variations, the one or more clinical recommendations may include notifying a clinician and enrolling in a clinical trial.

In some variations, the one or more clinical recommendations may include ordering one or more additional labs.

In some variations, the one or more additional labs may provide one or more clinical observations determined by at least identifying one or more similar patients and identifying the one or more clinical observations as a set of most important features included in a clinical data of the one or more similar patients but missing from the clinical data of the patient.

In some variations, the set of most important features may be determined by altering one or more input features provided to the first machine learning model to identify a set of input features that cause the risk score of the patient to exceed the first threshold, and ranking the set of input features based on a magnitude of change relative to a baseline value.

In some variations, a measured clinical outcome of the patient as a result of implementing the one or more clinical recommendations may be determined. An expected clinical outcome of the patient may be determined. An adjustment to one or more hyper-parameters associated with the determining of the one or more clinical recommendations may be determined based at least on a difference between the measured clinical outcome and the expected clinical outcome.

In some variations, the difference between the measured clinical outcome and the expected clinical outcome may be decomposed into a first fraction attributable to a change in clinical practice directly engendered by the one or more clinical recommendations and a second fraction attributable to other unmeasured confounders. The adjustment to the one or more hyper-parameters associated with the determining of the one or more clinical recommendations may be determined based at least on the first fraction attributable to the change in clinical practice directly engendered by the one or more clinical recommendations.

In some variations, the adjustment to the one or more hyper-parameters may be determined by at least performing a Bayesian optimization.

In some variations, the measured clinical outcome and the expected clinical outcome may include one or more of a mortality, a length of stay, and a cost of care.

In some variations, the expected outcome of the patient may be determined by applying a fourth machine learning model trained to predict clinical outcomes.

In some variations, the one or more hyper-parameters may include an alert threshold for various patient groups, a maximum number of allowable alarms per time period, and a frequency of ordering of additional labs.

In some variations, the adjustment to the one or more hyper-parameters may be determined for a specific subset of patients as defined based on one or more of phenotypes, care settings, and diagnostic related grouping (DRG).

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the stratification of sepsis patients, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts a system diagram illustrating an example deployment of a patient stratification system, in accordance with some example embodiments;

FIG. 1B depicts a block diagram illustrating an example of a patient stratification engine, in accordance with some example embodiments;

FIG. 1C depicts a schematic diagram illustrating another example deployment of a patient stratification engine, in accordance with some example embodiments;

FIG. 2A depicts a block diagram illustrating an example of a clinical data analyzer, in accordance with some example embodiments;

FIG. 2B depicts a block diagram illustrating an example of a process for generating a trust set for a clinical data analyzer, in accordance with some example embodiments;

FIG. 3 depicts a block diagram illustrating an example of a stratification controller, in accordance with some example embodiments;

FIG. 4 depicts a block diagram illustrating an example of a false positive network, in accordance with some example embodiments;

FIG. 5 depicts a block diagram illustrating an example of a false negative network, in accordance with some example embodiments;

FIG. 6A depicts a block diagram illustrating an example of a decision analyzer, in accordance with some example embodiments;

FIG. 6B depicts a block diagram illustrating an example of an uncertainty network (UncNet), in accordance with some example embodiments;

FIG. 7 depicts a flowchart illustrating an example of a process for machine learning enabled patient stratification; and

FIG. 8 depicts a block diagram illustrating an example of a computing system, in accordance with some example embodiments.

When practical, similar reference numbers denote similar structures, features, or elements.

### DETAILED DESCRIPTION

Early recognition of diseases and timely initiation of medical interventions have been shown to be an effective approach to improve clinical outcomes. The increased adoption of Electronic Health Records (EHRs) in hospitals has motivated the development of machine learning based models for the early prediction of physiological decompensation. However, a major barrier to implementation of such predictive system is the high false alarm rate, which can lead to high cognitive burden on the end-user (e.g., alarm fatigue) and unnecessary and potentially harmful interventions. This high false alarm rate may be due to a number of factors including, for example, uncertainty in the coefficients of the prediction model (e.g., machine learning model), data quality and characteristics (e.g., a shift in demographic characteristics), healthcare-specific variations in data generating process (e.g., frequency of ordering of laboratory tests), and/or the like. False negative diagnosis (e.g., a risk score below the detection threshold for a true case) may occur due to missing data. Meanwhile, false positive diagnosis (e.g., risk score above the detection threshold for a control case) may occur due to erroneous data or an outlier case previously unseen by the prediction model.

In some example embodiments, a patient stratification system may be configured to support a multi-pronged machine learning based patient stratification workflow. For example, the patient stratification system may include a data analyzer trained to detect non-conformal clinical data and a stratification controller configured to generate, based on conformal clinical data, one or more clinical recommendations based on the probability of false positives and false negatives associated with the conformal clinical data. The stratification controller may include a predictive model trained to determine a risk score based on features extracted from clinical data identified as conformal by the data analyzer. Furthermore, the stratification controller may include one or more additional models (e.g., a false positive network, a false negative network, an uncertainty network, and/or the like) trained to assess the validity of risk score determined by the predictive model (e.g., probabilities the risk score being a true positive or a true negative, uncertainty associated with the risk score, and/or the like) and before a decision analyzer generates one or more actionable clinical recommendations. For instance, the decision analyzer may combine information about data missingness and quality, metrics of target data distribution shift and/or conformity, model uncertainty, and/or other contextual information (e.g., patient, provider, and/or care facility-related information) to predict potential true or false episodes of an impending critical event and stratify patients into actionable sub-groups.

As one example deployment, the patient stratification system may be configured to diagnose and generate clinical recommendations for sepsis. Accordingly, the various models included in the patient stratification system may be trained using a development dataset that includes electronic health record (EHR) data of one million patients across multiple academic medical centers before the patient stratification system is deployed in a community hospital and exposed to a target dataset in which at least some patients exhibit novel genetic makeup and comorbidities not present in the development dataset. In this deployment environment, the patient stratification system is able to use features derived from the characteristics of data at the community hospital and metrics of model uncertainty to significantly reduce the incidence of false positives and false negative in its predictions. The resulting actionable recommendations provided by the patient stratification system (e.g., order additional labs to reduce model uncertainty, consult with a clinician to start the patient on antibiotics, and/or the like) is therefore associated with better overall clinical outcome as well.

In some example embodiments, the patient stratification system may implement a variety of techniques for reducing false negatives and false positives in the output of its constituent machine learning models. These techniques include combining information embedded in model-uncertainty, correlations among observations, and a shift in characteristics of target data distribution to significantly reduce false alarms and provide actionable recommendations. For example, the predictive model may rely on the observation that physiological systems operate under closed feedback loop and often multiple abnormal laboratory measurements occur in consort prior to patient decompensation. In some cases, data uncertainty may be captured by randomly excluding certain observations and assessing the change in predictive risk scores. Meanwhile, model uncertainty may be captured by systematically blocking certain nodes in the model (e.g., randomly ignoring or “dropping out” the output of some layers of a neural network) and assessing the change in predictive risk scores (e.g., Monte Carlo dropout). In such settings, a large variation in risk may indicate that the system is overly relying on a few spurious observations to produce a risk score. Accordingly, the stratification controller may use this information to identify potential false positives or false negatives.

In some example embodiments, the data analyzer of the patient stratification system may use a distance metric (e.g., Euclidean distance, cosine distance, and/or the like) to compare observations (or a representation of the data such as an encoding) from a target dataset to that of the development dataset to detect outliers and to characterize the degree of similarity or conformity of various datasets at deployment time. The patient stratification system may use this information, in association with other features of data missingness and uncertainty, to identify potential false positives or false negatives, and provide actionable recommendations to the end-user (e.g., order additional labs to reduce model uncertainty, consult with a clinician to start the patient on antibiotics, and/or the like).

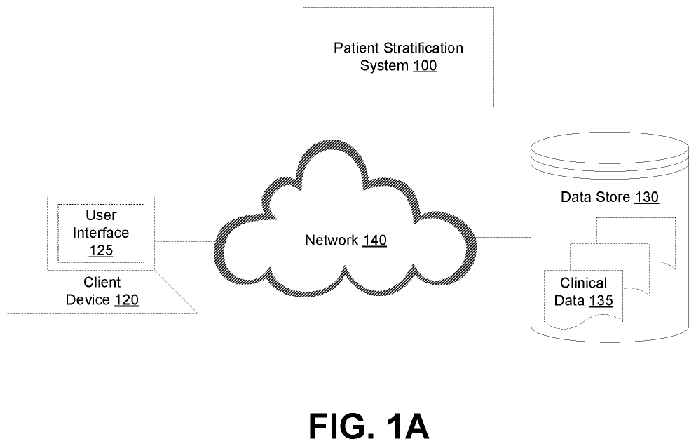

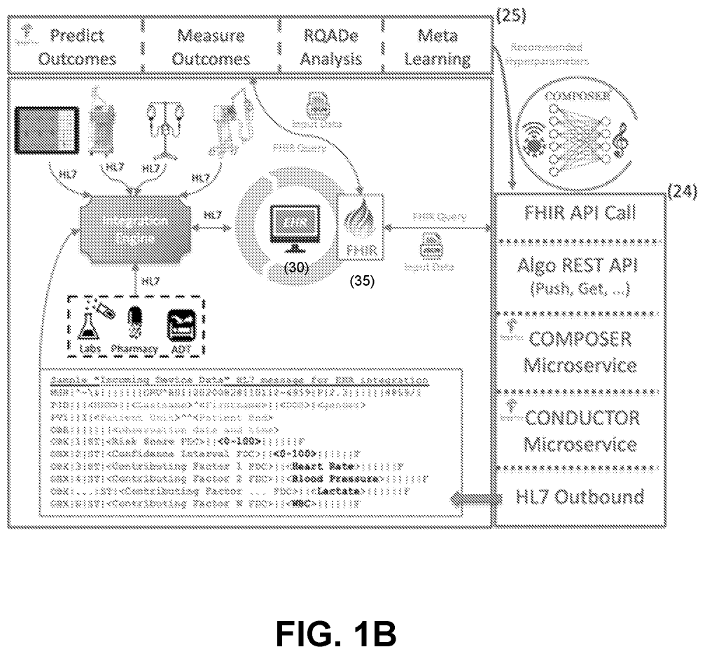

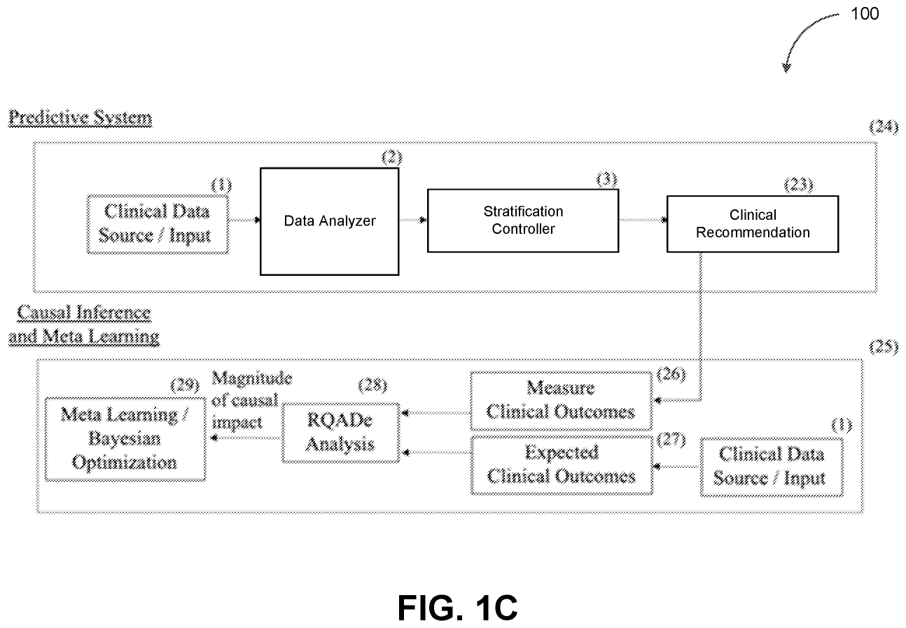

FIG. 1A depicts a system diagram illustrating an example deployment of a patient stratification system 100, in accordance with some example embodiments. Referring to FIG. 1A, the patient stratification system 100 may be communicatively coupled to a client device 120 and a data store 130 via a network 140. The client device 120 may be a processor-based device including, for example, a workstation, a desktop computer, a laptop computer, a smartphone, a tablet computer, a wearable apparatus, and/or the like. The data store 130 may be a database including, for example, a relational database, a graph database, an in-memory database, a non-SQL (NoSQL) database, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the patient stratification system 100 may be configured to determine, based at least on a patient's clinical data 135 (e.g., electronic health record (EHR) data) from the data store 130, one or more clinical recommendations for the patient. For example, the patient stratification system 100 may apply one or more machine learning models to determine the one or more clinical recommendations. Moreover, the one or more clinical recommendations may be determined based on the clinical data 135 as well as the uncertainties associated with the clinical data 135 and the machine learning models operating on the clinical data 135. Examples of clinical recommendations include notifying a clinician, enrolling in a clinical trial, and ordering additional labs. The one or more clinical recommendations may be display, for instance, in a user interface 125 at the client device 120.

Another example deployment of the patient stratification system 100 is shown in FIG. 1B. FIG. 1B shows an example of an end-to-end implementation of the patient stratification system 100 deployed in a clinical setting. As shown in FIG. 1B, the patient stratification system 100 may include a predictive sub-system (24) and a causal inference and meta learning sub-system (25). Moreover, as shown in FIG. 1B, the patient stratification system 100 may obtain clinical data by interacting with an electronic health record (EHR) system (30) via Fast Healthcare Interoperability Resources (FHIR) application programming interface (API) calls to a Fast Healthcare Interoperability Resources (FHIR) server (35). Moreover, FIG. 1B shows the patient stratification system 100 sending one or more clinical recommendations recommended scores back to the electronic health record (EHR) system (30) via an incoming device data interface using Health Level Seven (HL7) protocols.

FIG. 1C depicts a block diagram illustrating an example of the patient stratification system 100, in accordance with some example embodiments. Referring now to FIG. 1C, the predictive sub-system 24 of the patient stratification system 100 includes a clinical data source (1), a data analyzer (2), and a stratification controller (3). As shown in FIG. 1C, the output of the stratification controller (3) includes a clinical recommendation (23). In some cases, the clinical data source (1) may be configured to query one or more data sources, including a standardized data source the data store 130 shown in FIG. 1A and the Fast Healthcare Interoperability Resources (FHIR) server in FIG. 1C. Moreover, the data analyzer (2) and the stratification controller (3) may include one or more machine learning models including, for example, a convolutional neural network, a recurrent neural network, a regression model, an instance-based model, a regularization model, a decision tree, a random forest, a Bayesian model, a clustering model, an associative model, a deep learning model, a dimensionality reduction model, an ensemble model. and/or the like.

Referring again to FIG. 1C, the casual inference and meta learning (CaMeL) sub-system (25) of the patient stratification system 100 may be configured to provide feedback on the performance of the predictive sub-system (24), thus enabling an end-to-end optimization of the hyper-parameters of the predictive sub-system (24). Examples of hyper-parameters subject to optimization include alert thresholds for various patient groups, maximum number of allowable alarms per day, the frequency of ordering of additional labs, and/or the like. The overall flow of information through the predictive sub-system (24) and the causal inference and meta learning sub-system (25) includes the predictive sub-system (24) predicting potential true or false episodes of an impending critical event and stratifying patients into actionable sub-groups. Once the clinical recommendation (23) output by the predictive sub-system (24) is acted upon, the Real-time Quality Improvement Assessment and Decomposition (RQADe) analyzer (28) may determine the risk adjusted change in clinical outcomes of interest which may include, for example, the difference between measured clinical outcomes (26) and expected clinical outcomes (27). The resulting causal impact metric is then used in association with a Bayesian optimizer (29) to further fine-tune the predictive sub-system (24) hyper-parameters.

In some example embodiments, the causal inference and meta learning (CaMeL) sub-system (25) may track measured clinical outcomes (26), thus tracking improvements in clinical, quality, and, financial indices (e.g., hourly organ failure scores, mortality, length of stay, cost of care, compliance with recommended treatment protocols, and/or the like). As shown in FIG. 1C, the causal inference and meta learning sub-system (25) may also include models for predicting expected clinical outcomes (27). These may include, for example, a deep learning model trained to predict mortality, length of stay, cost of care, and/or the like. As will be described in more detail, patient outcomes may be computed based on the data representations of patient clinical data generated by an encoder.

The real-time quality improvement assessment and decomposition (RQADe) analyzer (28) shown in FIG. 1C may compare the measured clinical outcomes (26) and the expected clinical outcomes (27) to assess the impact of the clinical recommendations (23) generated by the decision analyzer (9). In some example embodiments, the real-time quality improvement assessment and decomposition (RQADe) analyzer (28) may track the cumulative difference (and the corresponding confidence intervals) between the observed clinical outcomes (26) and the expected clinical outcomes (27) to statistically assess whether deviations from expected clinical outcomes are attributable to the clinical recommendations (23) made by the decision analyzer (9). In some cases, the real-time quality improvement assessment and decomposition (RQADe) analyzer (28) may decompose the observed change in clinical outcomes (e.g., mortality) into a first fraction attributable to the change in clinical practice directly engendered by the clinical recommendations (23) output from the predictive sub-system (24), and a second fraction attributable to other unmeasured confounders (e.g., change in the patient case-mix and/or the like).

A meta learning engine (29) in the casual inference and meta learning sub-system (25) may perform Bayesian optimization to identify one or more changes in the hyper-parameters of the predictive sub-system (24) (e.g., alert thresholds, maximum number of allowable alarms per day, frequency of ordering of additional labs and/or the like) to maximize the impact of the clinical recommendations (23) output by the predictive sub-system (24) on the clinical outcome of interest. In some example embodiments, the meta learning engine (29) module may fine-tune the hyper-parameters of the predictive sub-system (24) for a specific subset of patients as defined, for example, by their phenotypes, care settings, diagnostic related grouping (DRG), and/or the like.

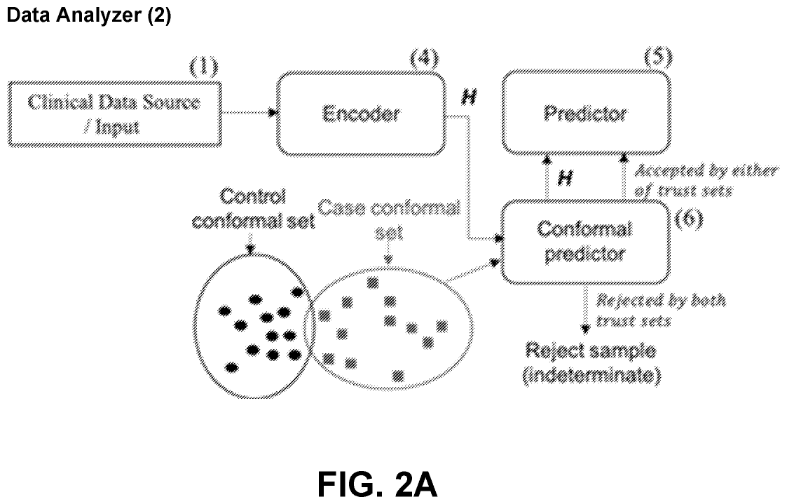

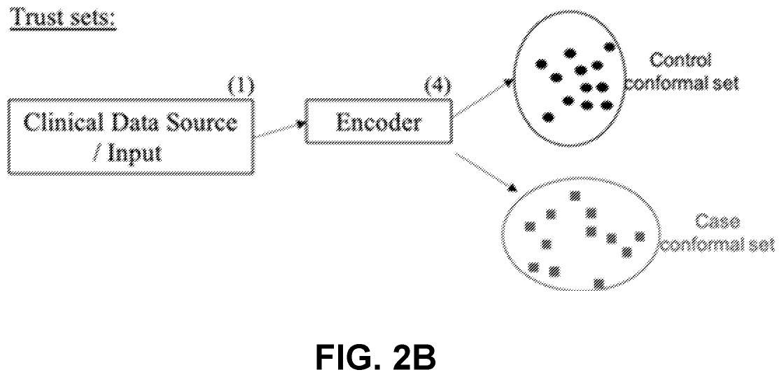

FIG. 2A depicts a block diagram illustrating an example of the data analyzer (2), in accordance with some example embodiments. In the example shown in FIG. 2A, the data analyzer (2) may include an encoder (4), a predictor (5), and a conformal predictor (6). In some cases, the encoder (4) may be implemented as a feed forward neural network. As shown in FIG. 2B, during a training phase, the encoder (4) may generate, based on a training set of clinical data from the clinical data source (1), two or more trust sets for evaluating patient clinical data by at least encoding the corresponding clinical data. The training set of clinical data used for generating the trust sets may correspond to a development dataset acquired from multiple academic medical centers. In some example embodiments, the two or more trusts sets can be constructed by at least clustering raw clinical data and/or encoded representations (e.g., raw observations or representations learned by the encoder (4)) of true cases (e.g., patients with sepsis), true controls (e.g., propensity matched patients without sepsis), and false positives (e.g., patients without sepsis but deemed septic during model training), and false negatives (e.g., patients with sepsis but deemed non-septic during model training). At inference time, the data analyzer (2) may use these trust sets to generate similarity and/or conformity metrics for a patient's clinical data, which are also used as features by the stratification controller (3) to determine the probability of false positives and false negatives associated with the patient's clinical data. In the example shown in FIG. 2B, the trust sets may include a control conformal set, which includes encoded representations of clinical data associated with patients without a particular disease (e.g., sepsis). Furthermore, the trust sets may include a case conformal set, which includes encoded representations of clinical data associated with patients with the particular disease (e.g., sepsis).

Referring back to FIG. 2A, at inference time, a patient's clinical data from the clinical data source (1) may be ingested by the encoder (4), which may reduce the dimensionality of the clinical data to generate a representation of the clinical data more suitable for prediction and similarity-based quantification. The conformal predictor (6) may compute, for the patient's clinical data, one or more similarity or conformity metrics indicative of how well the patient's clinical data aligns with the earlier generated trust sets (e.g., the case conformal set, the control conformal set, and/or the like). Whether the patient stratification system 100 is able to use the patient's clinical data to generate one or more reliable clinical recommendations (23) may be contingent on the similarity or conformity metrics of the clinical data satisfying one or more corresponding thresholds. For instance, where the conformal predictor (6) identifies the patient's clinical data as an outlier relative to the trust sets, the conformal predictor (6) may reject the patient's clinical data for further analysis by the patient stratification system 100. Alternatively, where the conformal predictor (6) determines that the patient's clinical data is consistent with the trust sets, the patient's clinical data may be passed to the predictor (5). The predictor (5), which may also be implemented as a feedforward neural network, may determine, based at least on the patient's clinical data, a risk score (e.g., a probability between 0 and 1) indicative of a diagnosis for the patient such as the patient's risk of physical decompensation.

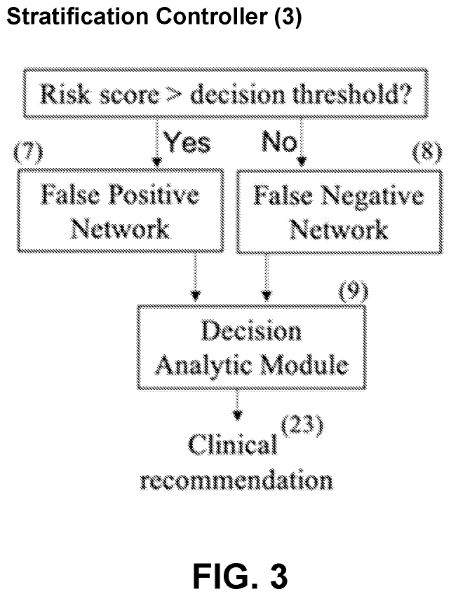

FIG. 3 depicts a block diagram illustrating an example of the stratification controller (3), in accordance with some example embodiments. As shown in FIG. 3, the stratification controller (3) includes a false positive network (7) and a false positive network (8). Depending on whether the risk score for the patient output by the data analyzer (2) satisfies a decision threshold, the stratification controller (3) may pass the patient's clinical data (e.g., from the clinical data source (1)) to either the false positive network (7) or the false negative network (8). For example, if the risk score is higher than the decision threshold, the conductor module (3) may utilize the false positive network (7) to determine if the patient's risk score indicates a potential false positive or a true positive. Similarly, if the risk score is lower than the decision threshold, the stratification controller (3) may utilize the false negative network (8) to determine if the patient's risk score indicates a potential false negative or true negative. As shown in FIG. 3, the output of the false positive network (7) or the false negative network (7) may be provided to a decision analyzer (9) to generate the clinical recommendation (23) for the patient.

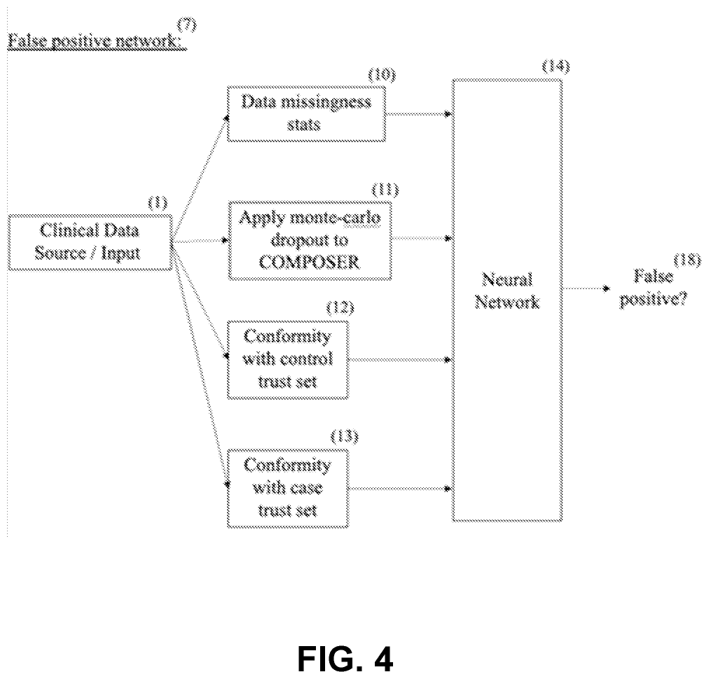

FIG. 4 depicts a block diagram illustrating an example of the false positive network (7), in accordance with some example embodiments. As shown in FIG. 4, the false positive network (7) may ingest a variety of contextual and analytically derived information to determine whether the patient's above-threshold risk score is likely to be a true positive. In some cases, the false positive network (7) may be configured to classify whether the patient's above-threshold risk score is a true positive or a false positive based on features derived from the patient's clinical data and the machine learning models implementing the data analyzer (2). For example, the false positive network (7) may ingest five categories of features including, for example, quantity (e.g., percentage) of missing clinical variables (10), uncertainty associated with the risk score (e.g., inter-quartile range) (11) obtained by applying Monte Carlo dropout to the machine learning models implementing the data analyzer (2), extent of conformity of patient's clinical data to the control conformal set (12), extent of conformity of the patient's clinical data to the case conformal set (13), and number of nearest neighbors with discordant labels or the spread in risk score via repeated substitution of all or selected number of missing features by the corresponding or most relevant feature values from the nearest neighbors, respectively. These features may be provided to the false positive network (7), which may be implemented as a neural network, to obtain an output indicating whether the patient's above-threshold risk score is a true positive or a false positive.

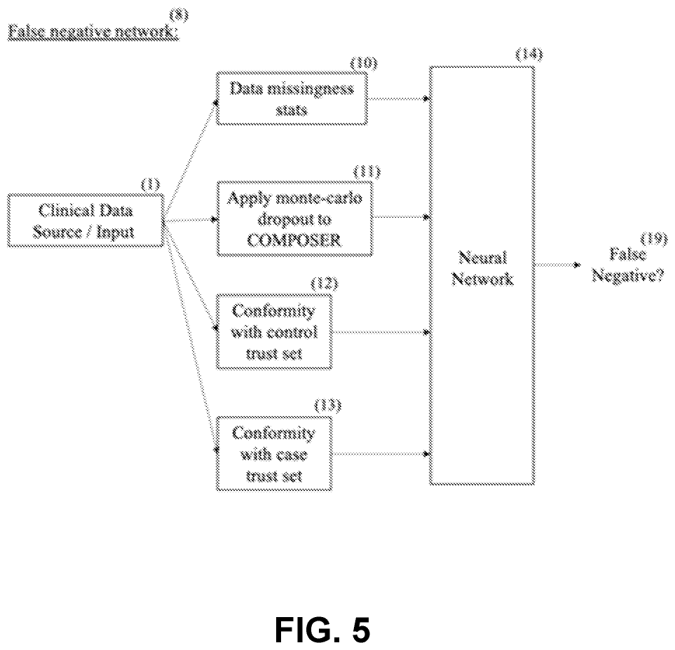

FIG. 5 depicts a block diagram illustrating an example of the false negative network (8), in accordance with some example embodiments. As shown in FIG. 5, the false negative network (8) may also ingest a variety of contextual and analytically derived information to determine whether a patient's below-threshold risk score is likely to be a false negative. For example, the false negative network (8) may be configured to classify whether the patient's below-threshold risk score is a true negative or a false negative based on features derived from the patient's clinical data and the machine learning models implementing the data analyzer (2). FIG. 5 shows five categories of features including, for example, quantity (e.g., percentage) of missing clinical variables (10), uncertainty of the patient's risk score uncertainty (e.g., inter-quartile range) (11) obtained by applying Monte Carlo dropout to the machine learning models implementing the data analyzer (3), extent of conformity of the patient's clinical data to the control conformal set (12), extent of conformity of the patient's clinical data to the case conformal set (13), and number of nearest neighbors with discordant labels or the spread in risk score via repeated substitution of all or selected number of missing features by the corresponding or most relevant feature values from the nearest neighbors, respectively. These features may be provided to the false negative network (8), which may be implemented as a neural network, to obtain an output indicating whether the patient's below-threshold risk score is a true negative or a false negative.

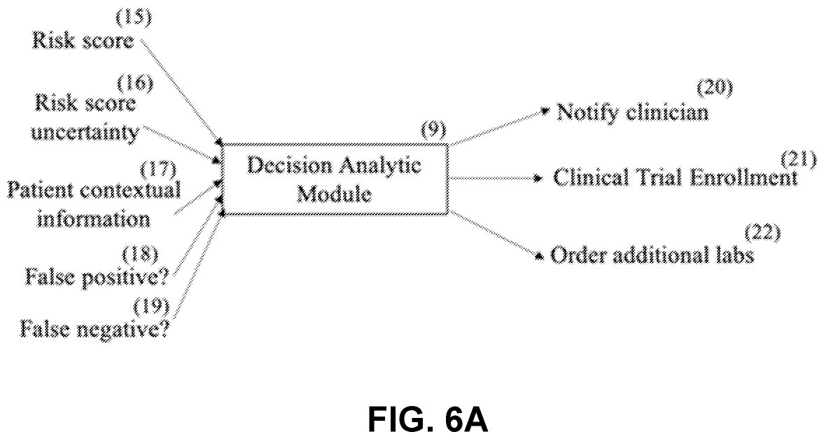

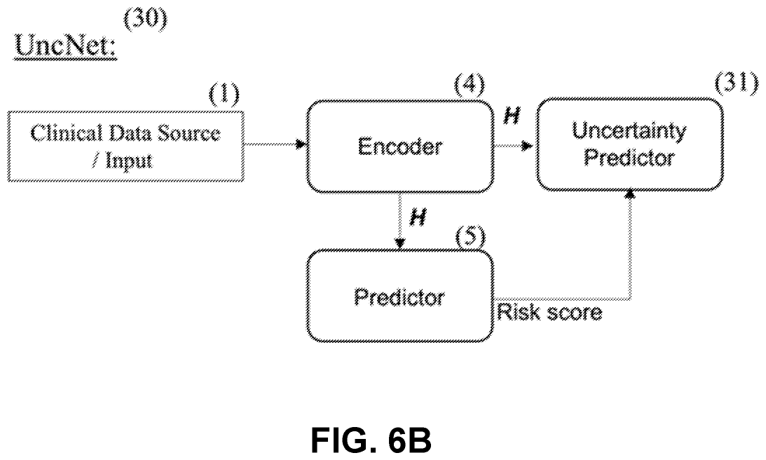

FIG. 6A depicts a block diagram illustrating an example of the decision analyzer (9), which may be a context aware (e.g., care level such as emergency, general wards, or intensive care) decision tree that combines information from the predictor (5), the false positive network (7), and the false negative network (8) to generate the clinical recommendation (23). As shown in FIG. 6A, inputs to the decision analyzer (9) may include the patient's risk score (15) from the predictor (5) of the data analyzer (2), the uncertainty associated with the risk score (16), patient contextual information (17) (e.g., level of care, presence of chronic illness, and/or the like), likelihood of the risk score being a false positive (18), and/or likelihood of the risk score being a false negative (19). Moreover, as shown in FIG. 6A, examples of the clinical recommendation (23) include notifying clinician (20), enrolling in a clinical trial (21), and ordering additional labs (22).

In some example embodiments, to determine the clinical recommendation (23) including the ordering of additional labs (22), the decision analyzer (9) may perform a cluster analysis, such as a k-nearest neighbour (KNN) search, using the representation of a patient's clinical data generated by the encoder (4) to identify one or more similar patients (e.g., a top k number of similar patients). Among these similar patients, the decision analyzer (9) may identify those with the lowest prediction uncertainty and determine the most important features (e.g., using a “feature importance” ranking technique) that are not missing from the clinical data of these patients but might have been missing the clinical data of the original patient. In this context, an important feature may be a clinical observation having a large effect on the risk score (15) output of the predictor (5) as well as the output of the decision analyzer (19). The decision analyzer (9) may determine to order additional labs (22) providing features in order to reduce the prediction uncertainty (16) and, by corollary, reduce the likelihood of a false positive (18) or false negative (19) associated with the risk score (15).

In some example embodiment, the decision analyzer (9) may identify the aforementioned most important features by systematically and iteratively altering the input features (e.g., using gradient descent) to change output of the predictor (5). For example, if the risk score (15) of the patient is 0.4 and the decision threshold applied by the stratification controller (3) is 0.5, a gradient descent approach may be applied in which the input features are altered to increase the risk score (15) above the 0.5 decision threshold. The features are then sorted according to the magnitude of their respective changes from the corresponding baseline values. The decision analyzer (9) (or some other logic unit) may use this information, in addition to information about the age of each feature, to determine which clinical observations are needed to improve model confidence. A set of features that are most likely to change the output of the decision analyzer (9) may be identified based on a ranking of the altered input features. Additional labs (22) providing the corresponding clinical observations may be ordered to reduce the prediction uncertainty of the decision analyzer (9) and, by corollary, reduce the likelihood of a false positive (18) or false negative (19) associated with the risk score (15).

FIG. 6B depicts a block diagram illustrating an example of an uncertainty network (UncNet) (30), in accordance with some example embodiments. In some example embodiments, the uncertainty network (30) may include an uncertainty predictor (31) configured to combine the output of the encoder (4) (e.g., a representation of the clinical data from the clinical data source (1)) and the corresponding risk score determined by the predictor (5) to determine an uncertainty associated with the risk score. As shown in FIG. 6A, in some cases, the uncertainty of the risk score computed for a patient may be used by the decision analyzer (9) to determine when, for instance, additional labs (22) may be required to reduce the prediction uncertainty and, by corollary, reduce the likelihood of a false positive or false negative associated with the risk score.

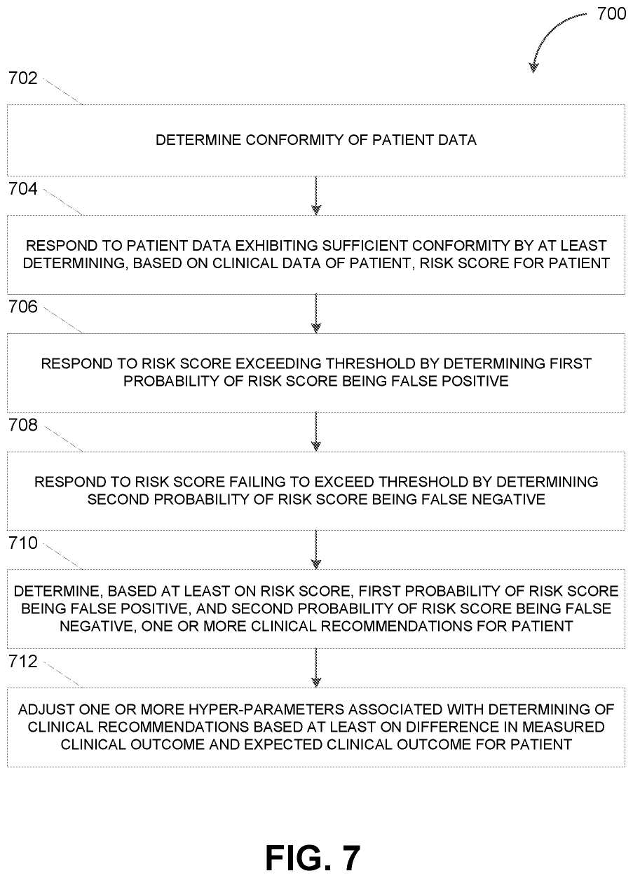

FIG. 7 depicts a flowchart illustrating an example of a process 700 for patient stratification, in accordance with some example embodiments. Referring to FIGS. 1A-C, 2A-B, 3-5, 6A-B, and 7, the process 700 may be performed by the patient stratification system 100 including, for example, the predictor sub-system (24) and the causal inference and meta learning sub-system (25).

At 702, the patient stratification system 100 may determine a conformity of a clinical data of a patient. In some example embodiments, the patient stratification system 100, for example, the data analyzer (2) of the predictor sub-system (24), may determine whether the clinical data of a patient from the clinical data source (1) exhibits sufficient conformity to one or more trust sets to support the generation of the clinical recommendation (23). For example, as shown in FIG. 2A, the conformal predictor encoder (6) may determine, based at least on a reduced dimension representation of the clinical data generated by the feed forward neural network implementing the encoder (4), whether the clinical data of the patient conforms to the control conformal set or the case conformal set.

At 704, the patient stratification system 100 may respond to the clinical data of the patient exhibiting sufficient conformity by at least determining, based at least on the clinical data of the patient, a risk score for the patient. In some example embodiments, where the clinical data of the patient is determined to exhibit sufficient conformity to the control conformal set or the case conformal set, the predictor (5) may determine, based at least on the clinical data of the patient, a risk score for the patient. In some cases, the risk score may be a probability (e.g., between 0 and 1) indicative of a diagnosis for the patient such as the patient's risk of physical decompensation. Contrastingly, where the clinical data of the patient is an outlier with respect to the control conformal set and the case conformal set, the data analyzer (2) may reject the clinical data for further analysis at least because the clinical data cannot support an accurate determination of the clinical recommendations (23).

At 706, the patient stratification system 100 may respond to the risk score exceeding a threshold by at least determining a first probability of the risk score being a false positive. In some example embodiments, the risk score of the patient output by the data analyzer (2) may be passed to the stratification controller (3) for further analysis. For example, as shown in FIG. 3, depending on whether the risk score for the patient output by the data analyzer (2) satisfies a decision threshold, the stratification controller (3) may pass the patient's clinical data (e.g., from the clinical data source (1)) to either the false positive network (7) or the false negative network (8). For example, if the risk score is higher than the decision threshold, the conductor module (3) may utilize the false positive network (7) to determine if the patient's risk score indicates a potential false positive or a true positive. As shown in FIG. 4, the false positive network (7) may make its determination based on five categories of features including, for example, quantity (e.g., percentage) of missing clinical variables (10), uncertainty associated with the risk score (e.g., inter-quartile range) (11) obtained by applying Monte Carlo dropout to the machine learning models implementing the data analyzer (2), extent of conformity of patient's clinical data to the control conformal set (12), extent of conformity of the patient's clinical data to the case conformal set (13), and number of nearest neighbors with discordant labels or the spread in risk score via repeated substitution of all or selected number of missing features by the corresponding or most relevant feature values from the nearest neighbors, respectively.

At 708, the patient stratification system 100 may respond to the risk score failing to exceed the threshold by at least determining a second probability of the risk score being a false negative. Alternatively, where the risk score of the patient does not exceed the decision threshold, the stratification controller (3) may utilize the false negative network (8) to determine if the patient's risk score indicates a potential false negative or true negative. As shown in FIG. 5, the false negative network (8) may make its determination based on four categories of features including, for example, quantity (e.g., percentage) of missing clinical variables (10), uncertainty of the patient's risk score uncertainty (e.g., inter-quartile range) (11) obtained by applying Monte Carlo dropout to the machine learning models implementing the data analyzer (3), extent of conformity of the patient's clinical data to the control conformal set (12), and extent of conformity of the patient's clinical data to the case conformal set (13).

At 710, the patient stratification system 100 may determine, based at least on the risk score, the first probability of the risk score being a false positive, and the second probability of the risk score being a false negative, one or more clinical recommendations for the patient. In some example embodiments, the decision analyzer (9) of the stratification controller (3) may be a context aware (e.g., care level such as emergency, general wards, or intensive care) decision tree that combines information from the predictor (5), the false positive network (7), and the false negative network (8) to generate the clinical recommendation (23). Examples of the clinical recommendation (23) include notifying clinician (20), enrolling in a clinical trial (21), and ordering additional labs (22).

At 712, the patient stratification system 100 may adjust one or more hyper-parameters associated with determining the one or more clinical recommendations based at least on a difference in a measured clinical outcome and an expected clinical outcome for the patient. In some example embodiments, the casual inference and meta learning (CaMeL) sub-system (25) of the patient stratification system 100 may be configured to provide feedback on the performance of the predictive sub-system (24). Dong so may enable an end-to-end optimization of the hyper-parameters of the predictive sub-system (24) including, for example, alert thresholds for various patient groups, maximum number of allowable alarms per time period, the frequency of ordering of additional labs, and/or the like. For example, as shown in FIG. 1C, once the clinical recommendation (23) output by the predictive sub-system (24) is acted upon, the Real-time Quality Improvement Assessment and Decomposition (RQADe) analyzer (28) may determine the risk adjusted change in clinical outcomes of interest (e.g., the difference between measured clinical outcomes (26) and expected clinical outcomes (27)). The resulting causal impact metric may be used in association with a Bayesian optimizer (29) to further fine-tune the predictive sub-system (24) hyper-parameters.

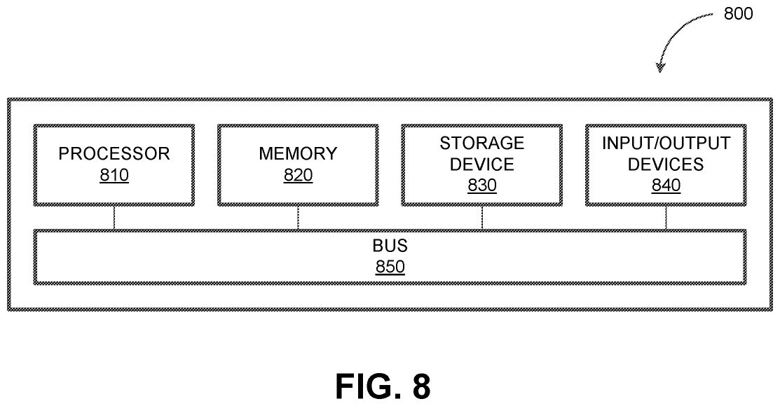

FIG. 8 depicts a block diagram illustrating an example of computing system 800, in accordance with some example embodiments. Referring to FIGS. 1A and 8, the computing system 800 may be used to implement the patient stratification system 100 and/or any components therein.

As shown in FIG. 8, the computing system 800 can include a processor 810, a memory 820, a storage device 830, and input/output devices 840. The processor 810, the memory 820, the storage device 830, and the input/output devices 840 can be interconnected via a system bus 850. The processor 810 is capable of processing instructions for execution within the computing system 800. Such executed instructions can implement one or more components of, for example, the patient stratification system 100 and/or the like. In some implementations of the current subject matter, the processor 810 can be a single-threaded processor. Alternately, the processor 810 can be a multi-threaded processor. The process may be a multi-core processor have a plurality or processors or a single core processor. The processor 810 is capable of processing instructions stored in the memory 820 and/or on the storage device 830 to display graphical information for a user interface provided via the input/output device 840. The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, or a tape device, or other suitable persistent storage means. The input/output device 840 provides input/output operations for the computing system 800. In some implementations of the current subject matter, the input/output device 840 includes a keyboard and/or pointing device. In various implementations, the input/output device 840 includes a display unit for displaying graphical user interfaces. According to some implementations of the current subject matter, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet)

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A system, comprising:
at least one data processor; and
at least one memory storing instructions, which when executed by the at least one data processor, result in operations comprising:
applying a first machine learning model to determine, based at least on a clinical data of a patient, a risk score for the patient;
in response to the risk score for the patient exceeding a first threshold, applying a second machine learning model to determine a first probability of the risk score being a false positive;
in response to the risk score for the patient failing to exceed the first threshold, applying a third machine learning model to determine a second probability of the risk score being a false negative; and
determining, based at least on the risk score, the first probability of the risk score being the false positive, and the second probability of the risk score being the false negative, one or more clinical recommendations for the patient.

2. The system of claim 1, wherein the operations further comprise:
determining a conformity metric indicative of a similarity between the clinical data of the patient and one or more conformal sets;
in response to the conformity metric satisfying a second threshold, applying the first machine learning model to determine the risk score for the patient; and
in response to the conformity metric failing to satisfy the second threshold, rejecting the clinical data of the patient as indeterminate.

3. The system of claim 2, wherein the operations further comprise:
encoding the clinical data of the patient to generate a reduced dimension representation of the clinical data; and
determining, based at least on the reduced dimension representation of the clinical data, the conformity metric indicative of the similarity between the clinical data of the patient and one or more conformal sets.

4. The system of claim 2, wherein the conformity metric comprises a Euclidean distance or a cosine distance.

5. The system of claim 2, wherein the one or more conformal sets include a control conformal set of clinical data associated with patients without a disease and a case conformal set of clinical data associated with patients with the disease.

6. The system of claim 2, wherein the one or more conformal sets are generated by at least clustering training data including true cases of patients with a disease, true controls of patients without the disease, false positives of patients without the disease but diagnosed as having the disease, and false negatives of patients with the disease but diagnosed as without the disease.

7. The system of claim 2, wherein the first probability of the risk score being the false positive and the second probability of the risk score being the false negative are determined based on one or more of a quantity of missing clinical variables in the clinical data, an uncertainty associated with the risk score, an extent of conformity between the clinical data and the one or more conformal sets, and a number of nearest neighbors with discordant labels or a spread in the risk score.

8. The system of claim 2, wherein the operations further comprise:
determining an uncertainty associated with the risk score of the patient; and
determining, based at least on the uncertainty associated with the risk score, the one or more clinical recommendations for the patient.

9. The system of claim 8, wherein the uncertainty associated with the risk score of the patient includes an uncertainty associated with the first machine learning model, and wherein the uncertainty associated with the first machine learning model is determined by at least applying a Monte Carlo dropout to assess a change in the risk score caused by ignoring an output of one or more layers of the first machine learning model.

10. The system of claim 8, wherein the uncertainty associated with the risk score of the patient includes an uncertainty associated with the clinical data, and wherein the uncertainty associated with the clinical data is determined by at least assessing a change in the risk score caused by excluding random portions of the clinical data.

11. The system of claim 8, wherein the uncertainty associated with the risk score of the patient is assessed based on a quantity similar patients with discordant labels and/or a spread in risk score determined at least by repeated substitution of at least one missing feature of the patient by a value of a corresponding feature or a most relevant feature from similar patients.

12. The system of claim 1, wherein the first machine learning model, the second machine learning model, and the third machine learning model comprise feed forward neural networks.

13. The system of claim 1, wherein the one or more clinical recommendations for the patient is determined by applying a decision tree to the risk score, an uncertainty associated with the risk score, a contextual information for the patient, the first probability of the risk score being the false positive, and the second probability of the risk score being the false negative.

14. The system of claim 1, wherein the one or more clinical recommendations are generated based at least on a context of the patient being one of emergency, general wards, or intensive care.

15. The system of claim 1, wherein the one or more clinical recommendations include notifying a clinician and enrolling in a clinical trial.

16. The system of claim 15, wherein the one or more clinical recommendations include ordering one or more additional labs.

17. The system of claim 16, wherein the one or more additional labs provide one or more clinical observations determined by at least identifying one or more similar patients and identifying the one or more clinical observations as a set of most important features included in a clinical data of the one or more similar patients but missing from the clinical data of the patient.

18. The system of claim 17, wherein the set of most important features is determined by altering one or more input features provided to the first machine learning model to identify a set of input features that cause the risk score of the patient to exceed the first threshold, and ranking the set of input features based on a magnitude of change relative to a baseline value.

19. The system of claim 1, wherein the operations further comprise:
determining a measured clinical outcome of the patient as a result of implementing the one or more clinical recommendations;
determining an expected clinical outcome of the patient; and
determining, based at least on a difference between the measured clinical outcome and the expected clinical outcome, an adjustment to one or more hyper-parameters associated with the determining of the one or more clinical recommendations.

20. The system of claim 19, wherein the operations further comprise:
decomposing the difference between the measured clinical outcome and the expected clinical outcome into a first fraction attributable to a change in clinical practice directly engendered by the one or more clinical recommendations and a second fraction attributable to other unmeasured confounders; and
determining, based at least on the first fraction attributable to the change in clinical practice directly engendered by the one or more clinical recommendations, the adjustment to the one or more hyper-parameters associated with the determining of the one or more clinical recommendations.

21-52. (canceled)
